(19) United States
(12) Patent Application Publication
(10) Pub. No.: US 2013/0158411 A1
(43) Pub. Date: Jun. 20, 2013

(54) ULTRASOUND DIAGNOSTIC APPARATUS AND CONTROL METHOD FOR ULTRASOUND DIAGNOSTIC APPARATUS

(71) Applicant: SEIKO EPSON CORPORATION, Tokyo (JP)

(72) Inventor: Mitsuru MIYASAKA, Shiojiri (JP)

(73) Assignee: SEIKO EPSON CORPORATION, Tokyo (JP)

(21) Appl. No.: 13/715,187

(22) Filed: Dec. 14, 2012

(30) Foreign Application Priority Data
Dec. 16, 2011 (JP) 2011-275359

(51) Int. Cl. A61B 8/00; A61B 8/08

(52) U.S. Cl. CPC A61B 8/429; A61B 8/4281; A61B 8/4444; A61B 8/54; A61B 8/08; A61B 8/0825
USPC 600/472

(57) ABSTRACT

Measuring is performed by bringing a gel pad GP which is an acoustic coupling medium into contact with a measuring portion which has concavities and convexities and by sending and receiving ultrasound from a probe via the gel pad GP. At this time, the contact pressure of the probe and the gel pad GP is detected using pressure sensors which are provided in the probe (first pressure sensor and second pressure sensor). Then, ultrasound focusing is performed using the detected pressure of the pressure sensors.

### CROSS-REFERENCE TO RELATED APPLICATIONS

This application claims priority to Japanese Patent Application No. 2011-275359 filed on Dec. 16, 2011. The entire disclosure of Japanese Patent Application No. 2011-275359 is hereby incorporated herein by reference.

### BACKGROUND

1. Technical Field

The present invention relates to an apparatus, which performs ultrasound diagnostics using ultrasound, and the like.

2. Background Technology

In ultrasound diagnostic apparatuses in the related art, a focusing technique is being used in order to match the focus (focal point) with a position which is a measuring target (referred to below as “measuring target position”). For example, an electron focus technique is disclosed in PTL1 which changes the focus distance or focus direction by changing delay time of a pulse signal which is input to a plurality of ultrasound transducers provided in a probe.

Japanese Laid-open Patent Publication No. H9-108223 (Patent Document 1) is an example of the related art.

### SUMMARY

### Problems to be Solved by the Invention

In a case of ultrasound testing of a portion where there are convexities and concavities such as a thyroid or a breast, a method is used where measuring is performed by placing an acoustic coupling medium above the body surface and by sending and receiving ultrasound from above via the acoustic coupling medium without the probe being in direct contact with the body surface. As the acoustic coupling medium, for example, an acoustic coupling polymer gel is known.

The acoustic coupling polymer gel described above is formed as a soft viscoelastic body in order to be excellent at conforming to the shape of the body surface. As a result, there can be an adverse effect with regard to the ultrasound diagnostic apparatus due to the acoustic coupling polymer gel easily changing shape depending on the pressing force of the probe. That is, since the distance from the transmission source of the ultrasound to the measuring target position changes, there can be a problem in that the measuring target position deviates from the focus position which has been set and an image of a measuring target position is not able to be suitably obtained.

The invention has been realized in consideration of the problems described above and has an advantage of proposing a novel method for appropriately performing control of focusing in an ultrasound diagnostic apparatus which performs measuring by sending and receiving ultrasound via an acoustic coupling medium.

### Means Used to Solve the Above-Mentioned Problems

A first embodiment for solving the problems described above is an ultrasound diagnostic apparatus which performs measuring by bringing an acoustic coupling medium into contact with a measuring portion which has concavities and convexities and by sending and receiving ultrasound via the acoustic coupling medium and the ultrasound diagnostic apparatus is provided with a probe which sends and receives ultrasound, a pressure sensor which is provided in the probe which measures the contact pressure between the probe and the acoustic coupling medium, and a focusing control section which controls either of transmission focusing or reception focusing of the ultrasound (referred to below as “focusing”) using the detected pressure of the pressure sensor.

According to the first embodiment, measuring is performed by bringing the acoustic coupling medium into contact with the measuring portion which has concavities and convexities and by sending and receiving ultrasound via the acoustic coupling medium. The acoustic coupling medium is able to change shape due to the pressing operation of the probe, and the contact pressure between the probe and the acoustic coupling medium is measured using the pressure sensor which is provided in the probe. Since the focusing of the ultrasound is controlled using the detected pressure, it is possible to appropriately control the focusing even in a case where the thickness of the acoustic coupling medium changes depending on the contact pressure with the probe.

In this case, as another embodiment, there is a control method for an ultrasound diagnostic apparatus which performs measuring by bringing an acoustic coupling medium into contact with a measuring portion which has concavities and convexities and by sending and receiving ultrasound via the acoustic coupling medium, and due to configuring of the control method for the ultrasound diagnostic apparatus to include measuring the contact pressure between a probe and the acoustic coupling medium and controlling focusing of the ultrasound using the contact pressure, it is possible to obtain the same action effects as the first embodiment.

In addition, as a second embodiment, in the ultrasound diagnostic apparatus of the first embodiment, the ultrasound diagnostic apparatus can be configured so that the focusing control section has a focus distance control section which controls focus distance based on the size of the detected pressure.

According to the second embodiment, it is possible to match the focus to the measuring target position even in a case where the distance to the measuring target position has changed by controlling the focus distance based on the size of the detected pressure of the pressure sensor.

In this case, as a third embodiment, in the ultrasound diagnostic apparatus of the second embodiment, it is preferable that the ultrasound diagnostic apparatus be configured so that the focus distance control section control controls the focus distance to be shortened as the detected pressure is increased.

The distance from the probe to the measuring target position is shortened due to the thickness of the acoustic coupling medium being reduced when the acoustic coupling medium is pressed strongly by the probe. Therefore, it is possible for the focus distance control section to realize appropriate focus control by shortened the focus distance as the detected pressure of the pressure sensor is increased as in the third embodiment.

In addition, as the fourth embodiment, in the ultrasound diagnostic apparatus of any of the first to the third embodiments, the ultrasound diagnostic apparatus can be configured to be further provided with a storage section which stores profile data which sets a focusing control parameter with regard to the detected pressure, and the focusing control section controls the focusing based on the control parameter which corresponds to the detected pressure.

According to the fourth embodiment, the storage section stores the profile data which sets the focusing control parameter with regard to the detected pressure. Then, focusing control is made easy by the focusing control section controlling the focusing based on the control parameter which corresponds to the detected pressure.

In addition, as the fifth embodiment, in the ultrasound diagnostic apparatus of any of the first to the fourth embodiments, the ultrasound diagnostic apparatus can be configured to be provided with a plurality of the pressure sensors and to be further provided with a pressure gradient calculation section which calculates the pressure gradient of the contact pressure at the contact surface with the probe based on the detected pressure of the plurality of pressure sensors, and the focusing control section controls the focusing using the pressure gradient.

According to the fifth embodiment, the probe is provided with the plurality of pressure sensors. For example, there are cases where the probe is tilted with regard to a measuring portion in practice even when a doctor or a practitioner intends to press the probe so as to be perpendicular with regard to the measuring portion. Therefore, the pressure gradient calculation section calculates the pressure gradient of the contact pressure at the contact surface with the probe based on the detected pressure of the plurality of pressure sensors. Then, it is possible to realize appropriate focus control by the focusing control section controlling the focusing using the pressure gradient.

In addition, as the sixth embodiment, in the ultrasound diagnostic apparatus of the fifth embodiment, it is preferable that the ultrasound diagnostic apparatus be configured so that the focusing control section has a focus direction control section which controls focus direction using the pressure gradient.

According to the sixth embodiment, it is possible to match the focus to the measuring target position even in a case where the probe is tilted with regard to the measuring portion by the focus direction control section controlling the focus direction using the pressure gradient.

### BRIEF DESCRIPTION OF THE DRAWINGS

Referring now to the attached drawings which form a part of this original disclosure:

FIG. 1A is a schematic configuration diagram of a probe and FIG. 1B is a usage state diagram of a probe;

FIG. 2 is a functional configuration diagram of an ultrasound diagnostic apparatus of a first embodiment;

FIG. 3 is a data configuration diagram of profile data;

FIG. 4 is a flow chart illustrating the flow of a first focusing control process;

FIG. 5A is a functional configuration diagram of a processing section of a second embodiment and FIG. 5B is a data configuration diagram of a storage section of the second embodiment;

FIG. 6 is an explanatory diagram of an estimation method of a tilting direction and a tilting angle of a probe; and

FIG. 7 is a flow chart illustrating the flow of a second focusing control process.

### DETAILED DESCRIPTION OF EXEMPLARY EMBODIMENTS

### 1. FIRST EMBODIMENT

### 1-1. Probe Configuration

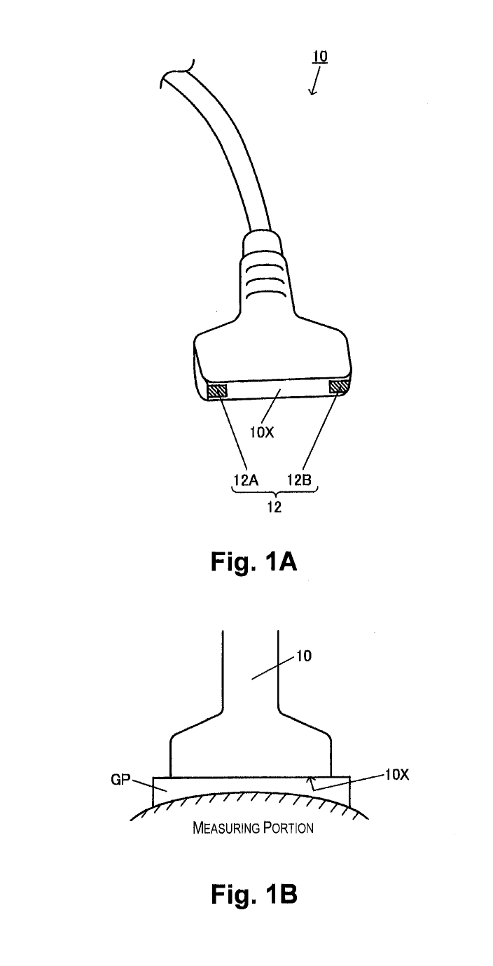

FIG. 1A is a diagram illustrating a schematic configuration of an ultrasound probe 10 (referred to below simply as “probe”) in the embodiment. The probe 10 is an apparatus which sends and receives ultrasound and has a built-in ultrasound oscillation unit for sending and receiving ultrasound to and from a contact surface 10X. Since usage by the contact surface 10X being brought into contact with a measuring portion is a typical example, the probe 10 can also be referred to as a contacting unit or a type thereof which sends and receives ultrasound. There are also cases of being called a searching unit.

In the embodiment, a case is assumed where measuring is performed by a pad (referred to below as “gel pad”) GP, which is formed by an acoustic coupling polymer gel which is a type of acoustic coupling medium, being brought into contact with a measuring portion where there are convexities and concavities such as a thyroid or a breast and ultrasound being sent and received via the gel pad. The acoustic coupling polymer gel is a transparent soft viscoelastic body, was developed with the object of carrying out ultrasound testing of a surface layer portion where there are convexities and concavities with higher accuracy, and is commonly known.

As one characteristic configuration of the embodiment, a plurality of pressure sensors 12 are provided at the contact surface 10X of the probe 10. Specifically, a first pressure sensor 12A is provided at one edge section and a second pressure sensor 12B is provided at the other edge section of the contact surface 10X in the longitudinal direction. In the embodiment, focusing of the ultrasound from the probe 10 is controlled using detected pressure of the pressure sensors 12.

FIG. 1B is a diagram illustrating a usage state of the probe 10. A doctor or a practitioner places the gel pad GP at the measuring portion where there are convexities and concavities and pressurizes the measuring portion by pressing with the contact surface 10X of the probe from above. In this state, an ultrasound diagnostic apparatus 1 generates a diagnostic image of the measuring target position by sending and receiving ultrasound from the ultrasound oscillation unit which is provided in the probe 10.

### 1-2. Functional Configuration

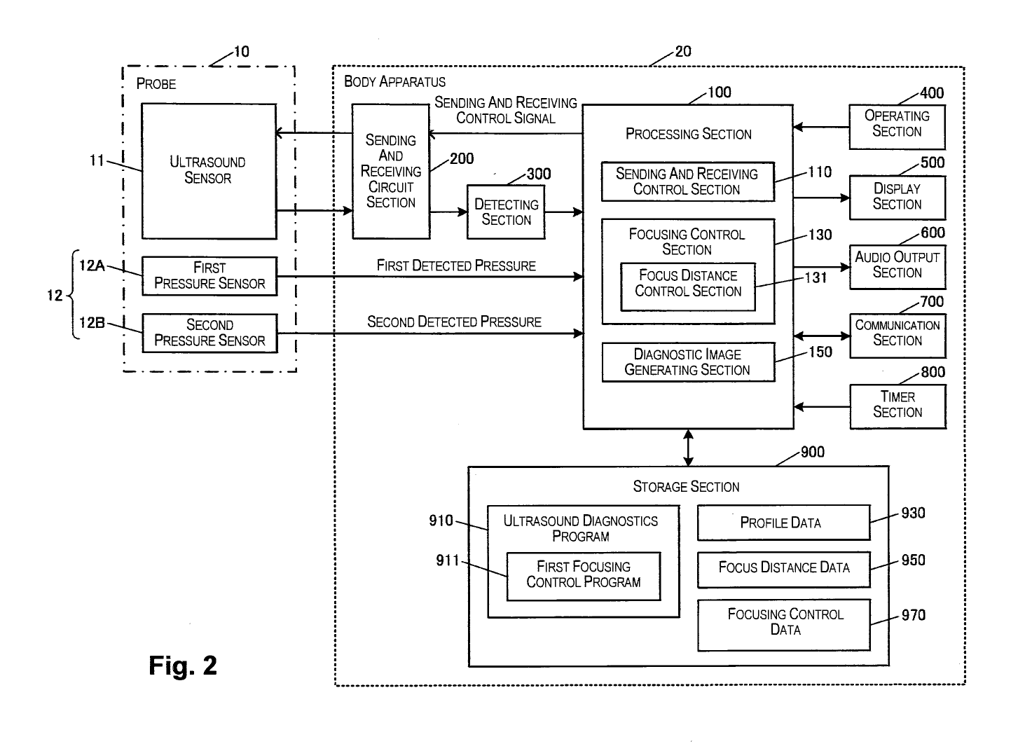

FIG. 2 is a block diagram illustrating an example of a functional configuration of the ultrasound diagnostic apparatus 1 of the embodiment. The ultrasound diagnostic apparatus 1 is provided with the probe 10 and a body apparatus 20.

The probe 10 is configured to have an ultrasound sensor 11 and the pressure sensors 12 (the first pressure sensor 12A and the second pressure sensor 12B).

The ultrasound sensor 11 is a sensor which irradiates an ultrasound pulse signal to the measuring portion and receives the reflected waves and is configured to have an ultrasound oscillation unit array where a plurality of ultrasound oscillation units are lined up in an array formation.

The pressure sensor 12 detects pressure which the contact surface 10X receives from the gel pad in a state where the contact surface 10X is in contact with the gel pad. It is possible to use semiconductor pressure sensors, electrostatic capacitance pressure sensors, or the like as the pressure sensors 12 and it is possible to mount the pressure sensors 12 to the contact surface 10X of the probe 10 by, for example, being processed into a sheet shape.

The body apparatus 20 is configured to be provided with a processing section 100, a sending and receiving circuit section 200, a detecting section 300, an operating section 400, a display section 500, an audio output section 600, a communication section 700, a timer section 800, and a storage section 900 as the main functional sections.

The sending and receiving circuit section 200 is a sending and receiving circuit which switches between an ultrasound transmission mode and an ultrasound reception mode using a time division method according to a trigger signal which is output from a sending and receiving control section 110.

The sending and receiving circuit section 200 is configured to have a pulse generating circuit (pulsar) which generates a pulse signal with a predetermined frequency, a transmission delay circuit which delays the signal pulse, and the like as a configuration for transmission. In addition, the sending and receiving circuit section 200 is configured to have an amplifier which amplifies a reception signal, a filter which extracts a predetermined frequency component from the reception signal, an A/D converter which converts the reception signal with an analog format into a digital signal, a reception delay circuit which delays the reception signal, and the like as a configuration for reception.

The sending and receiving circuit section 200 generates pulse signals while receiving trigger signals from the sending and receiving control section 110 with a timing which is synchronized with the trigger signals and outputs the pulse signals to each of the ultrasound oscillation units with a delay according to delay time which is set by a focusing control section 130. Due to this, the transmission waves are temporarily delayed with regard to the ultrasound oscillation units which are lined up in an array formation and focus distance and focus direction of the ultrasound is controlled. This is equivalent to ultrasound transmission focusing.

On the other hand, ultrasound reception signals which are output from the ultrasound oscillation units are received while the trigger signals are not being received from the sending and receiving control section 110 and are output to the processing section 100 after the predetermined frequency component is attenuated by delaying the reception signal according to the delay time which is set by the focusing control section 130. This is equivalent to ultrasound reception focusing.

In the embodiment, the beam forming and the focusing (transmission focusing and reception focusing) of the ultrasound signal is realized by the setting of the delay time with regard to each of the ultrasound oscillation units being performed as a digital signal process using software.

The detecting section 300 detects the reception signal of an ultrasound echo (RF signal) which is output from the sending and receiving circuit section 200. The detecting section 300 is configured to have a logarithm detecting circuit which performs logarithmic compression and amplitude envelope detection with regard to the reflected wave of the ultrasound.

The processing section 100 is a control apparatus and a computation apparatus which comprehensively controls each section of the ultrasound diagnostic apparatus 1 and is configured to have, for example, a microprocessor such as a CPU (Central Processing Unit) or a DSP (Digital Signal Processer), an ASIC (Application Specific Integrated Circuit), and the like.

The processing section 100 has the sending and receiving control section 110, the focusing control section 130, and a diagnostic image generating section 150 as the main functional sections. However, the functional sections are only a description of one example of an applied example and not all of the functional sections are necessarily essential configuration units.

The sending and receiving control section 110 controls the sending and receiving of the ultrasound from the probe 10. Specifically, a sending and receiving control signal is output in order to periodically switch the mode of the ultrasound sensor 11 between a transmission mode where an ultrasound pulse signal is transmitted and a reception mode where an ultrasound echo signal is received.

The focusing control section 130 controls the ultrasound focusing. In the first embodiment, the focusing control section 130 has a focus distance control section 131 as a functional section. The focus distance control section 131 controls the focus distance based on the size of the detected pressure of the first pressure sensor 12A and the second pressure sensor 12B.

Profile data 930, which sets a focusing control parameter with regard to the detected pressure of the pressure sensor 12, is stored in the storage section 900. The focus distance control section 131 controls the focus distance using the profile data 930 and the detected pressures of the first pressure sensor 12A and the second pressure sensor 12B.

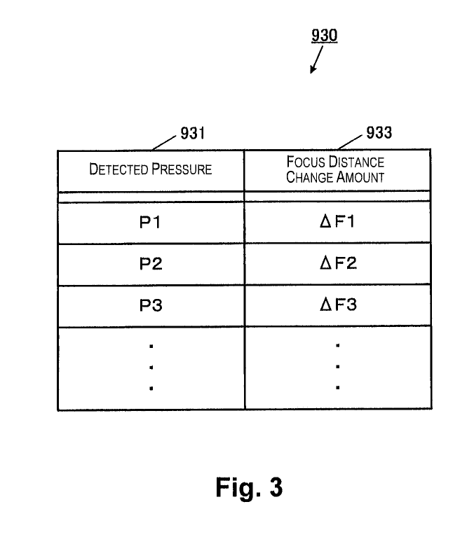

FIG. 3 is a diagram illustrating an example of the data configuration of the profile data 930. Detected pressures 931 of the pressure sensors 12 and focus distance change amounts 933 are stored so as to correspond in the profile data 930. That is, the amount of change in the focus distance according to the detected pressure of the pressure sensors 12 is stored as a control parameter value in the profile data 930. More specifically, a larger focus distance change amount is set as the detected pressure increases.

For example, the focus distance control section 131 averages the detected pressures of the first pressure sensor 12A and the second pressure sensor 1213 and reads out a focus distance change amount 933, which corresponds to the detected pressure 931 which is an average value, from the profile data 930. Then, the focus distance is updated by subtracting the focus distance change amount 933, which has been read out, from the focus distance which was set as an initial setting. When the gel pad is strongly pressed by the probe 10, the distance from the ultrasound oscillation units to the measuring target position is shortened by the gel pad being significantly depressed. As a result, the focus distance is shortened as the detected pressure of the pressure sensors 12 increases.

It is possible to realize the control of the focus distance by setting the delay times with regard to each of the ultrasound oscillation unit which are provided in the probe 10. The delay times, which are set with regard to each of the ultrasound oscillation units, are formulated using a known computation formula where, for example, the focus distance and the ultrasound beam irradiation angle are the variables. In the first embodiment, the delay times are computed and set for each of the ultrasound oscillation units by substituting the focus distance which is determined based on the detected pressure of the first pressure sensor 12A and the detected pressure of the second pressure sensor 12B into the computation formula. Due to this, the controlling of the focusing distance is realized.

Here, there are the transmission focusing and the reception focusing in the ultrasound focusing but either one method or both methods can be adopted. The sending and receiving circuit section 200 of the embodiment switches between the ultrasonic transmission mode and the ultrasound reception mode using a time division method. As a result, the transmission focusing and the reception focusing are controlled by controlling the timing when the ultrasound is transmitted and the timing when the reflected waves of the ultrasound are received using the transmission mode and the reception mode.

The diagnostic image generating section 150 generates a diagnostic image of the measuring target position based on the reception signal of the reflected waves of the ultrasound which is detected using the detecting section 300. It is possible to generate an image with various formats such as an A mode image, a B mode image, or an M mode image as the diagnostic image.

The operating section 400 is an input apparatus which is configured to have a keyboard, a button switch, and the like which are operated by a doctor or a practitioner.

The display section 500 is a display apparatus which is configured to have an LCD (Liquid Crystal Display) or the like and performs various types of display based on a display signal which is input from the processing section 100. A diagnostic image, which is generated by the diagnostic image generating section 150, or the like is displayed in the display section 500.

The audio output section 600 is an audio output apparatus which is configured to have a speaker or the like and performs various types of audio output based on an audio output signal which is input from the processing section 100.

The communication section 700 is a communication apparatus for sending and receiving information which is used in the apparatus to and from an external information processing apparatus such as a personal computer (PC) in accordance with the control of the processing section 100. As the communication method of the communications section 700, it is possible to apply various formats such as a format where a cable which complies with a predetermined communication standard is connected in a wired manner or a format where wireless communication is performed using short-distance wireless communication.

The timer section 800 is a timer apparatus which is configured to have a crystal oscillator, which is formed by a crystal resonator and an oscillator circuit, or the like and measures time. The time measuring of the timer section 800 is output at any time to the processing section 100.

The storage section 900 is configured to have a storage apparatus such as a ROM (Read Only Memory), a flash ROM, a RAM (Random Access Memory), or the like. The storage section 900 stores a system program of the ultrasound diagnostic apparatus 1, a program for realizing each of the functional sections of the sending and receiving control function and the focusing control function, data, and the like. In addition, there is a work area which temporarily stores processing data of various types of processing, processing results, and the like.

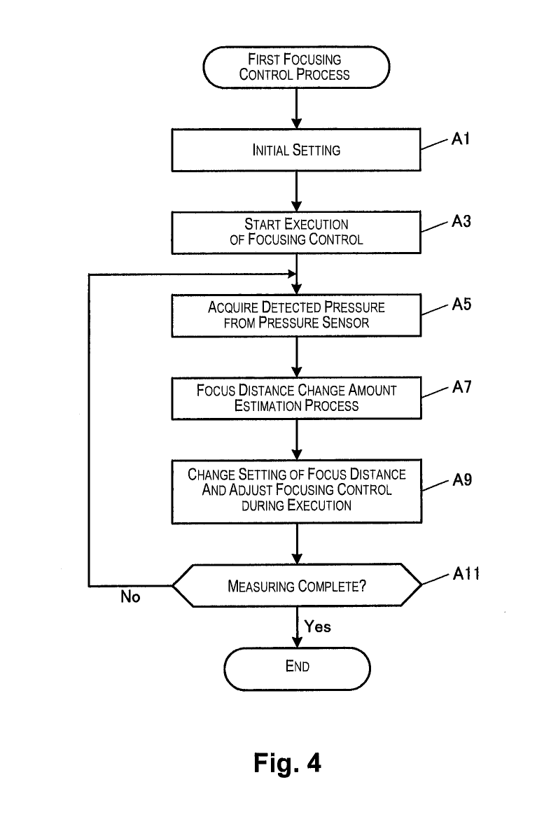

For example, an ultrasound diagnostics program 910, which is read out by the processing section 100 and is realized as ultrasound diagnostics processing, is stored in the storage section 900 as a program. The ultrasound diagnostics program 910 includes a first focusing control program 911 which is executed as a first focusing control process (refer to FIG. 4) as a sub routine.

In addition, the profile data 930 (refer to FIG. 3), focus distance data 950, and focusing control data 970 are stored in the storage section 900 as data.

The focus distance data 950 is data on the focus distance which is used when the focus distance control section 131 sets the delay time, and as described above, is updated at any time according to the size of the detected pressure of the first pressure sensor 12A and the second pressure sensor 12B.

The focusing control data 970 is data which is used for the focusing control section 130 to realize control of the ultrasound focusing and the data on the delay time which is set with regard to each of the ultrasound oscillation units is included in this.

### 1-3. Process Flow

FIG. 4 is a flow chart illustrating the flow of a first focusing control process which is executed in accordance with the first focusing control program 911 which is stored in the storage section 900.

To begin with, the focus distance control section 131 initially sets the focus distance and the focus direction up to the measuring target position based on an operation signal from the operation section 400 (step A1). Then, the focusing control section 130 starts the execution of the focusing control based on the initial settings.

Next, the processing section 100 acquires the detected pressures from the first pressure sensor 12A and the second pressure sensor 1213 (step A5). Then, the processing section 100 performs a focus distance change amount estimation process (step A7). Specifically, the detected pressures which are acquired from the first pressure sensor 12A and the second pressure sensor 12B are averaged and the focus distance change amount is estimated by reading out the focus distance change amount 933 which corresponds to the detected pressures 931 which is an average value from the profile data 930 in the storage section 900.

After that, the focus distance control section 131 changes the setting of the focus distance based on the focus distance change amount which has been estimated and the focusing control which is being executed is adjusted (step A9). Specifically, the focus distance is changed using the focus distance change amount which was estimated in step A7 and sets the delay time with regard to each of the ultrasound oscillation units. Then, the pulse signal which is output with regard to each of the ultrasound oscillation units is controlled by the sending and receiving circuit section 200 so as to be delayed according to the delay time which has been set.

Next, the processing section 100 determines whether the measuring is complete (step A11) and the process returns to step A5 in a case where it has been determined that the measuring is not complete (step A11; No). In addition, the first focusing control process is completed in a case where it has been determined that the measuring is complete (step A11; Yes).

### 1-4. Action Effect

According to the first embodiment, in the ultrasound diagnostic apparatus 1, the measuring is performed by the gel pad which is the acoustic coupling medium is brought into contact with the measuring portion which has concavities and convexities and by sending and receiving ultrasound from the probe 10 via the gel pad. At this time, the contact pressure between the probe 10 and the gel pad is detected using the pressure sensors 12 (the first pressure sensor 12A and the second pressure sensor 12B) which are provided in the probe 10. Then, the focusing control section 130 performs the ultrasound focusing using the detected pressure of the pressure sensors 12.

Specifically, the focus distance control section 131 controls the focus distance based on the size of the detected pressure of the first pressure sensor 12A and the second pressure sensor 12B. At this time, due to the focus distance control section 131 reducing the focus distance as the detected pressure increases, it is possible for the focus to be appropriately matched to the measuring target position even in a case where the thickness of the gel pad is reduced (depressed) due to the probe 10 strongly pressing on the gel pad.

In addition, the profile data 930 is stored so that the detected pressures of the pressure sensors 12 and the focusing distance change amount correspond, and the focus distance is changed based on the focus distance change amount which corresponds to the detected pressures of the first pressure sensor 12A and the second pressure sensor 12B. Due to this, it is possible to simply perform the control of the focus distance.

### 2. SECOND EMBODIMENT

The second embodiment is an embodiment where the control of the focus direction is performed in addition to the control of the focus distance which was described by the first embodiment as the ultrasound focusing control. Here, repetitive descriptions are omitted by giving the same reference numbers with regard to the same configurations and the same steps in the flow chart of the first embodiment and the portion which is different to the first embodiment will be the center of the description.

Since the gel pad is a soft viscoelastic body, there are cases where the probe 10 is tilted with regard to the measuring portion in practice even when a doctor or a practitioner intends to press the probe 10 so as to be perpendicular with regard to the measuring portion. In this case, there are cases where the focus deviates from the measuring target position due to the ultrasound beam being irradiated in a diagonal direction. Therefore, in the second embodiment, a pressure gradient of the contact pressure at the contact surface 10X of the probe 10 is calculated based on the detected pressures of the first pressure sensor 12A and the second pressure sensor 12B. Then, the focus direction is controlled using the pressure gradient which has been calculated.

### 2-1. Functional Configuration

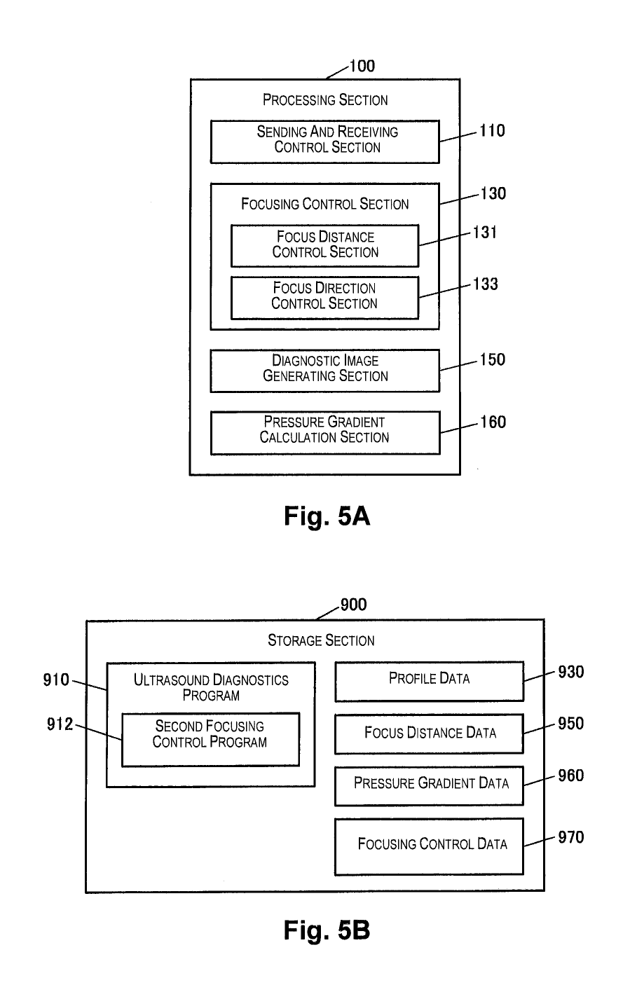

FIG. 5A is a diagram illustrating an example of functional sections of the processing section 100 in the second embodiment. The processing section 100 has the sending and receiving control section 110, the focusing control section 130, the diagnostic image generating section 150, and a pressure gradient calculation section 160. In addition, the focusing control section 130 has the focus distance control section 131 and a focus direction control section 133.

The pressure gradient calculation section 160 calculates the pressure gradient of the contact pressure at the contact surface 10X of the probe 10 based on the detected pressures of the first pressure sensor 12A and the second pressure sensor 12B which are provided in the probe 10. In the embodiment, the pressure gradient calculation section 160 calculates a difference between the detected pressure of the first pressure sensor 12A and the detected pressure of the second pressure sensor 12B (referred to below as “detected pressure difference”) as the pressure gradient.

The focus direction control section 133 controls the focus direction of the ultrasound based on the detected pressure difference which is calculated by the pressure gradient calculation section 160. Specifically, a tilting angle is estimated from the normal direction of the contact surface 10X based on the detected pressure difference.

As shown in FIG. 1, the pressure sensors 12 are respectively disposed at each of both edge sections of the contact surface 10X. As a result, the tilting angle which is able to be estimated in the embodiment is represented in a plane, which is perpendicular with the contact surface 10X and passes through detection positions of the first pressure sensor 12A and the second pressure sensor 12B, by an angle which is formed by the normal direction of the contact surface 10X and an arbitrary direction on the plane. Here, the angle is positive or negative and the normal direction of the contact surface 10X is set as a tilting angle “θ=0 degrees”.

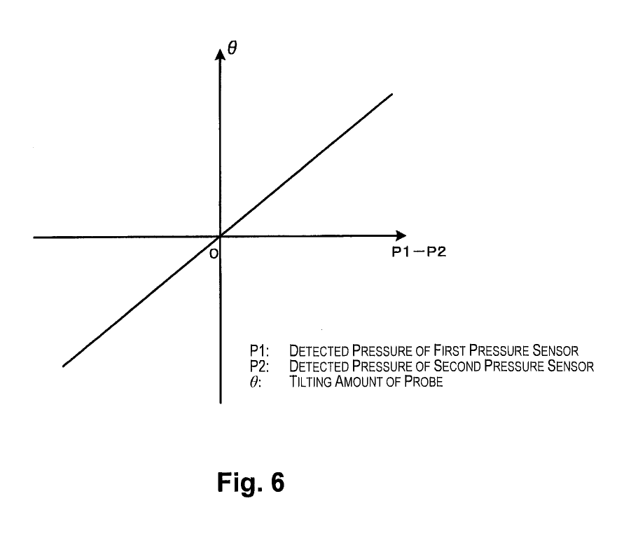

FIG. 6 is an explanatory diagram of an estimation method of the tilting angle of the probe 10. The detected pressure of the first pressure sensor 12A is set as “P1” and the detected pressure of the second pressure sensor 12B is set as “P2”. The sign of the tilting angle “θ” in a case where “P1” is larger than “P2” is set as positive and the sign of the tilting angle “θ” in a case where “P2” is larger than “P1” is set as negative. In this case, for example, the tilting angle “θ” of the probe 10 is estimated based on a linear function so that the tilting angle “θ” is larger as the detected pressure difference “P1−P2” increases.

It is possible for the control of the focus direction to be realized by setting the delay time with regard to each of the ultrasound oscillation units which are provided in the probe 10 in the same manner as the first embodiment. As described above, the delay time which is set with regard to each of the ultrasound oscillation units is estimated using, for example, a known computation formula where the focus distance and the irradiation angle of the ultrasound beam are variables. In the second embodiment, the delay times are individually set for each of the ultrasound oscillation units by determining the focus distance and the irradiation angle of the ultrasound beam based on the detected pressures of the pressure sensors 12 and substituting into the computation formula. Due to this, the control of the ultrasound focusing is realized.

In a state where there is a pressure gradient, the positional relationship of the contact surface 10X of the probe 10 and the measuring target position is not perpendicular and is tilted. Therefore, the delay times are set so that the ultrasound beam is tilted by the tilting angle “θ” which has been estimated so that the ultrasound beam is irradiated with regard to the measuring target position. That is, the irradiation angle (focus direction) of the ultrasound beam which is used in the computation of the delay times is adjusted based on the tilting angle of the probe 10 which has been estimated as described above.

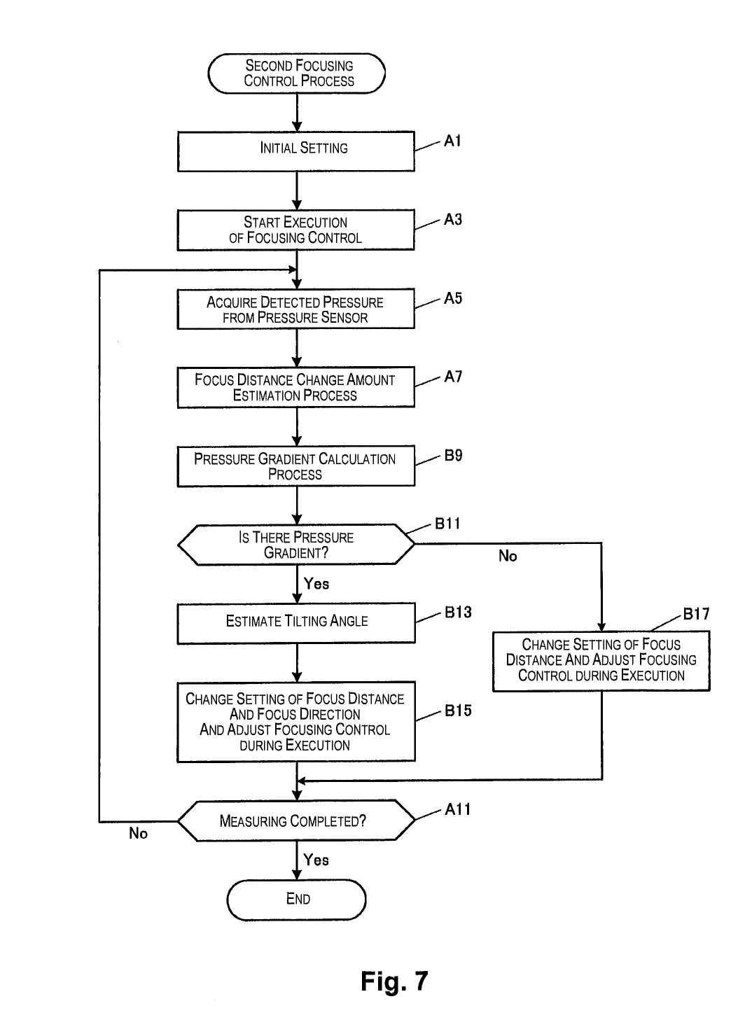

FIG. 5B is a diagram illustrating an example of a data configuration of the storage section 900 of the second embodiment. An ultrasound diagnostic program 910 is stored as a program in the storage section 900. The ultrasound diagnostic program 910 stores a second focusing control program 912 which is executed as a second focusing control process (refer to FIG. 7).

In addition, the profile data 930, the focus distance data 950, and pressure gradient data 960, and the focusing control data 970 are stored in the storage section 900 as data. Pressure gradients which are calculated using the pressure gradient calculation section 160 are stored in the pressure gradient data 960.

### 2-2. Process Flow

FIG. 7 is a flow chart illustrating the flow of the second focusing control process where the processing section 100 executes in accordance with the second focusing control program 912 which is stored in the storage section 900.

After step A7, the pressure gradient calculation section 160 performs a pressure gradient calculation process (step 139). Specifically, for example, the detected pressure difference “P1−P2” of the detected pressure “P1” of the first pressure sensor 12A and the detected pressure “P2” of the second pressure sensor 1213 is calculated as the pressure gradient.

Next, the processing section 100 determines whether there is a pressure gradient (step B11). Specifically, for example, it is determined whether the detected pressure difference “P1−P2” which is calculated in step B9 exceeds a predetermined threshold “θ”. In a case where it is determined that there is a pressure gradient (step B11; Yes), the focus direction control section 133 estimates a tilting angle of the probe 10 (step B13).

Then, the focus distance control section 131 and the focus direction control section 133 set and change the focus distance and the focus direction based on the focus distance change amount which is estimated in step A7 and the tilting angle which is estimated in step B13 and the focusing controlling during execution is adjusted (step B15). Then, the processing section 100 moves the process to step A11.

On the other hand, in a case where it is determined in B11 that there is no pressure gradient (step B11; No), the focus distance control section 131 sets and changes the focus distance based on the focus distance change amount which is estimated in step A7 and the focusing controlling during execution is adjusted (step B17). Then, the processing section 100 moves the process to step A11.

### 2-3. Action Effect

According to the second embodiment, the pressure gradient calculation section 160 calculates the pressure gradient of the contact pressure of the contact surface 10X of the probe 10 based on the detected pressure of the first pressure sensor 12A and the second pressure sensor 12B. Then, the focus direction control section 133 controls the focus direction using the pressure gradient which is calculated by the pressure gradient calculation section 160.

By calculating the gradient of the detected pressure of the first pressure sensor 12A and the second pressure sensor 12B which are provided at the edge sections of the contact surface 10X, it is possible to estimate the tilting angle of the probe 10. Then, by setting the delay time with regard to each of the ultrasound oscillation units based on the tilting angle which has been estimated, it is possible to appropriately match the focus to the measuring target position even in a case where the probe 10 is tilted with regard to the measuring position.

### 3. MODIFIED EXAMPLE

The invention is not limited to the embodiments described above and it is natural that appropriate modifications are possible within a range which does not depart from the gist of the present invention. Below, modified examples are described.

### 3-1. Pressure Sensor

In the embodiments described above, the case where the two pressure sensors 12 (the first pressure sensor 12A and the second pressure sensor 12B) are provided in the probe 10 is described as an example, but it is possible for the number of pressure sensors and the position where the pressure sensors are disposed to be appropriately changed.

For example, the pressure sensor 12 can be provided only at one location at substantially a central position in the contact surface of the probe 10. In this case, for example, the setting position of the pressure sensor 12 can be provided toward a side section of the probe 10 so as to not positionally overlap with a passing passage of the ultrasound.

In addition, the pressure sensors 12 can be provided at a total of three locations of left and right edge sections and a central position in the contact surface of the probe 10. In this case, the detected pressure of the pressure sensor 12 which is provided at the central position can be used for performing control of the focus distance, and the detected pressures of the pressure sensors 12 which are provided at the left and right edge positions can be used for performing control of the focus direction.

In addition, the pressure sensors 12 can be provided at four locations in the four corners of the contact surface 10X. In this case, it is possible to estimate the tilting angle on two axes (the angle around the axis in the longitudinal direction and the angle around the axis in the latitudinal direction of the contact surface 10X) with the normal direction of the contact surface 10X as a reference. The focus direction is adjusted based on the estimation result.

### 3-2. Switching Using Switch

In ultrasound testing, in order to generate a diagnostic image in a case where the measuring target position is seen from a different angle, there are cases where a doctor or a practitioner intentionally presses the probe 10 diagonally with regard to the measuring portion. Therefore, there can be a configuration where a switch of whether control of the focus direction is performed is provided without there being a configuration where control of the focus direction is normally performed as described in the second embodiment.

### 3-3. Profile Data

The profile data 930, which sets the focusing control parameter with regard to the detected pressures of the pressure sensors, can be stored in the storage section 900 for the different types of acoustic coupling media. The depression conditions of the acoustic coupling media in the case where the same pressing force is applied differs due to the types of the acoustic coupling media (for example, different thicknesses and different types of materials). As a result, it is preferable for the profile data 930 where the detected pressures of the pressure sensors 12 and the focus distance change amount correspond are set and selected in the initial setting for different types of acoustic coupling media.

### 3-4. Estimation of Tilting Angle

In the embodiments described above, the correlation characteristics of the detected pressure difference “P1−P2” of the two pressure sensors and the tilting angle “θ” of the probe 10 are approximated using a linear function but this is only one example. For example, the tilting angle “θ” can be estimated based on the correlation characteristics where, for example, the correlation characteristics are approximated using a non-linear function so that the tilting angle “θ” increases exponentially as the detected pressure difference “P1−P2” increases.

The entire disclosure of Japanese Patent Application No. 2011-275359, filed Dec. 16, 2011 is expressly incorporated by reference herein.

What is claimed is:

1. An ultrasound diagnostic apparatus which performs measuring by bringing an acoustic coupling medium into contact with a measuring portion which has concavities and convexities and by sending and receiving ultrasound via the acoustic coupling medium, comprising:
a probe which sends and receives ultrasound;
a pressure sensor which is provided in the probe which measures the contact pressure between the probe and the acoustic coupling medium; and
a focusing control section which controls either of transmission focusing or reception focusing of the ultrasound (referred to below as “focusing”) using the detected pressure of the pressure sensor.

2. The ultrasound diagnostic apparatus according to claim 1,
wherein the focusing control section has a focus distance control section which controls focus distance based on the size of the detected pressure.

3. The ultrasound diagnostic apparatus according to claim 2,
wherein the focus distance control section controls so that the focus distance is shortened as the detected pressure increases.

4. The ultrasound diagnostic apparatus according to claim 1, further comprising
a storage section which stores profile data which sets a focusing control parameter with regard to the detected pressure,
wherein the focusing control section controls focusing based on the control parameter which corresponds to the detected pressure.

5. The ultrasound diagnostic apparatus according to claim 1, further comprising
a plurality of the pressure sensors; and
a pressure gradient calculation section which calculates the pressure gradient of the contact pressure at the contact surface with the probe based on the detected pressures of the plurality of pressure sensors,
wherein the focusing control section controls the focusing using the pressure gradient.

6. The ultrasound diagnostic apparatus according to claim 5,
wherein the focusing control section has a focus direction control section which controls focus direction using the pressure gradient.

7. A control method for an ultrasound diagnostic apparatus, which performs measuring by bringing an acoustic coupling medium into contact with a measuring portion which has concavities and convexities and by sending and receiving ultrasound via the acoustic coupling medium, the method comprising:
measuring the contact pressure between a probe and the acoustic coupling medium; and
controlling focusing of the ultrasound using the contact pressure.
